(19) United States
(12) Patent Application Publication
(10) Pub. No.: US 2009/0005569 A1
(43) Pub. Date: Jan. 1, 2009

(54) PROCESS FOR THE PREPARATION OF 4-TRIFLUOROMETHYL-2(1H)-PYRIDINONE

(75) Inventors: Douglas C. Bland, Midland, MI (US); John Davies, Midland, MI (US)

Correspondence Address: DOW AGROSCIENCES LLC, 9330 ZIONSVILLE RD, INDIANAPOLIS, IN, 46268, US

(73) Assignee: Dow AgroSciences LLS, Indianapolis, IN (US)

(21) Appl. No.: 12/147,973

(22) Filed: Jun. 27, 2008

Related U.S. Application Data
(60) Provisional application No. 60/937,799, filed on Jun. 29, 2007.

(51) Int. Cl. C07D 211/02

(52) U.S. Cl. 546/249

(57) ABSTRACT

4-Trifluoromethyl-2(1H)-pyridinone is prepared from an alkyl vinyl ether and trifluoroacetyl chloride in a four step process.

This application claims the benefit of U.S. Provisional Application Ser. No. 60/937,799 filed on Jun. 29, 2007. The present invention concerns an improved process for the preparation of 4-trifluoromethyl-2(1H)-pyridinone.

### BACKGROUND OF THE INVENTION

U.S. Pat. No. 6,130,335 describes certain substituted pyridine-sulfonamide compounds and their use as herbicides. 4-Trifluoromethyl-2(1H)-pyridinone is a useful intermediate for the preparation of certain of these herbicides. 4-Alkoxy-1,1,1-trifluoro-3-buten-2-ones and 4,4-dialkoxy-1,1,1-trifluorobutan-2-ones are useful intermediates for preparing 4-trifluoromethyl-2(1H)-pyridinone; see, for example, U.S. Patent Publication 2005/0288511. Unfortunately, 4-alkoxy-1,1,1-trifluoro-3-buten-2-ones and 4,4-dialkoxy-1,1,1-trifluorobutan-2-ones are relatively expensive and somewhat unstable, i.e., it is recommended that they be stored under refrigeration. It would be desirable to have a process for preparing 4-trifluoromethyl-2(1H)-pyridinone in which the 4-alkoxy-1,1,1-trifluoro-3-buten-2-ones could be avoided and the 4,4-dialkoxy-1,1,1-trifluorobutan-2-ones be generated in situ, thus eliminating the transportation and storage issues associated with these raw materials.

### SUMMARY OF THE INVENTION

The present invention concerns an improved process for the preparation of 4-trifluoromethyl-2(1H)-pyridinone. More particularly, the present invention concerns a process for the preparation of 4-trifluoromethyl-2(1H)-pyridinone (I),

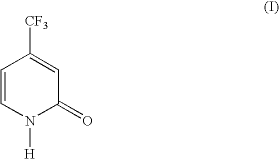

which comprises:
i) contacting an alkyl vinyl ether of the formula

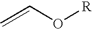

in which R represents a C₁-C₄ alkyl
   with trifluoroacetyl chloride to provide a 4-chloro-4-alkoxy-1,1,1-trifluoro-2-butanone of the formula:

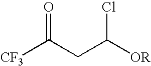

in which R is as previously defined;
ii) contacting the 4-chloro-4-alkoxy-1,1,1-trifluoro-2-butanone with a C₁-C₄ alcohol, to provide a mixture of acetals of the formula

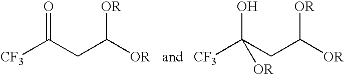

in which each R is independently as previously defined;
iii) contacting the mixture of acetals with a trialkyl phosphonoacetate of the formula:

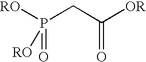

in which R is as previously defined,
   in the presence of a base and an alcohol or glycol solvent to provide a mixture of condensation products of the formula

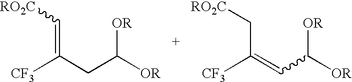

in which each R is independently as previously defined; and
iv) cyclizing the mixture of condensation products to provide 4-trifluoromethyl-2(1H)-pyridinone.

The mixture of condensation products may be cyclized to 4-trifluoromethyl-2(1H)-pyridinone by:
(a) reacting with an ammonium salt of an organic acid and/or with a mineral acid and/or with formamide.

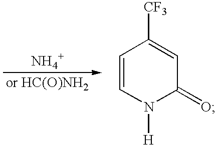

(b) reacting with an unsubstituted amide and methoxide to form a mixture of amides which is subsequently cyclized with acid

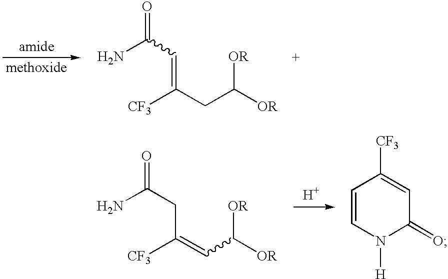

(c) reacting with an acid and water to form the lactone which is subsequently reacted with ammonia

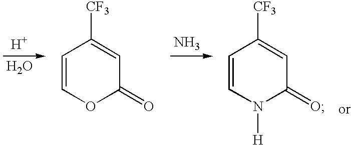

(d) reacting with an organic anhydride and pyridinium p-toluenesulfonate (PPTS) to form a mixture of dienoates which is subsequently cyclized by reacting with an ammonium salt of an organic acid (R′ represents alkyl or aryl) or a mineral acid or with formamide as in (a) or by reacting with an unsubstituted amide and methoxide to form a mixture of amides which is subsequently cyclized with acid as in (b).

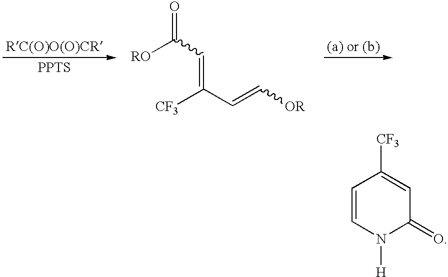

### DETAILED DESCRIPTION OF THE INVENTION

4-Trifluoromethyl-2(1H)-pyridinone (I) can also be represented by its tautomer, 4-trifluoromethyl-2-pyridinol (II).

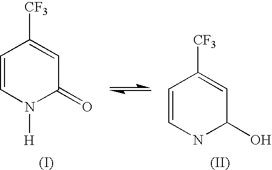

Unless specifically limited otherwise, the term “alkyl”, as used herein, includes within its scope straight chain, branched chain and cyclic moieties.

In the first step of the present invention, 4-chloro-4-alkoxy-1,1,1-trifluoro-2-butanones of the formula:

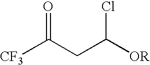

in which R represents a C₁-C₄ alkyl, are prepared by reacting an alkyl vinyl ether of the formula

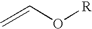

in which R represents a C₁-C₄ alkyl, with trifluoroacetyl chloride.

Approximately equimolar quantities of alkyl vinyl ether and trifluoroacetyl chloride are generally used in the process, although excesses of one or the other may be employed. In practice, a 10-50 percent stoichiometric excess of alkyl vinyl ether is often preferred.

The reaction is conducted either in the absence of a solvent, e.g., with excess alkyl vinyl ether, or in the presence of an anhydrous organic solvent. Preferred solvents are hydrocarbon solvents, most preferably aromatic hydrocarbons such as toluene.

The reaction is conducted at a temperature from about −10° C. to about 35° C. Temperatures from about 0° C. to about 20° C. are usually preferred.

In a typical reaction, the trifluoroacetyl chloride is bubbled below the surface of the alkyl vinyl ether, either neat or in the presence of a hydrocarbon solvent, between 0-5° C. The reaction is allowed to warm with stirring for about 1 hour, keeping the temperature no higher than room temperature. The crude reaction mixture containing the 4-chloro-4-alkoxy-1,1,1-trifluoro-2-butanone is usually used as is without further isolation or purification of the reaction mixture.

In the second step of the present invention, a mixture of acetals of the formula

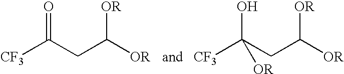

in which each R is independently as previously defined, are prepared by reacting the 4-chloro-4-alkoxy-1,1,1-trifluoro-2-butanone with a C₁-C₄ alcohol.

While equimolar quantities of 4-chloro-4-alkoxy-1,1,1-trifluoro-2-butanone and a C₁-C₄ alcohol are theoretically required, it is preferred to use an excess of the alcohol. In practice, methanol is most preferably used as the solvent.

The reaction is conducted at a temperature from about 0° C. to about 35° C. Temperatures from about 5° C. to about 25° C. are usually preferred.

In a typical reaction, the C₁-C₄ alcohol is cooled to between 0-5° C. and the crude reaction mixture containing the 4-chloro-4-alkoxy-1,1,1-trifluoro-2-butanone is con-added. The reaction is allowed to warm with stirring for about 3 hours, keeping the temperature no higher than room temperature. The crude reaction mixture containing the mixture of acetals is usually used as is without further isolation or purification of the reaction mixture.

In the third step of the present invention, the mixture of acetals of the formula

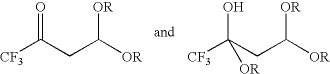

in which each R independently represents a C₁-C₄ alkyl is reacted with a trialkyl phosphonoacetate of the formula

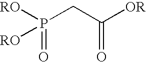

to provide a mixture of condensation products that result from reaction of 1 mole of acetal with 1 mole of phosphonoacetate, followed by loss of dialkyl phosphate. These condensation products can exist in a variety of isomeric forms. Typical of the mixture are compounds of the formula:

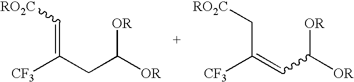

in which each R independently represents a C₁-C₄ alkyl, with CH₃ and CH₂CH₃ groups being preferred.

Approximately equimolar quantities of the mixture of acetals and trialkyl phosphonoacetate are generally used in the process, although excesses of one or the other may be employed.

The reaction of the mixture of acetals with trialkyl phosphonoacetate is conducted in the presence of a C₁-C₄ alkoxide. A stoichiometric amount of base is required, though an excess of base, e.g., from a 10 to a 50 percent excess, may be used.

The reaction of the third step is conducted in the presence of an alcohol or glycol solvent, particularly C₁-C₄ alcohols and glycols. Preferred solvents are methanol, ethanol and ethylene glycol and the solvents are preferably anhydrous. For purposes of recovery and recycle, it is most preferable for the alkoxide base and the alcohol solvent to be derived from the same C₁-C₄ alkyl group as employed in the mixture of acetals starting material and the trialkyl phosphonoacetate reactant. Optionally an aromatic hydrocarbon co-solvent such as toluene may be employed.

The reaction of the third step is conducted at a temperature from about −40° C. to about 60° C. Temperatures from about −10° C. to about 30° C. are usually preferred.

The product mixture is isolated by conventional techniques such as by extraction followed by evaporation of the solvent.

In a typical reaction, the mixture of acetals is prepared in an alcohol solvent and the trialkyl phosphonoacetate is added. After cooling to about 0° C., the mixture is slowly treated with the appropriate amount of alkali metal alkoxide in alcohol while maintaining the temperature at about 0-10° C. After the reaction is complete, the solvent is evaporated from the reaction mixture and the residue is dissolved in an organic solvent in which the inorganic components are insoluble, such as heptane or a similar alkane, toluene or methylene chloride. The organic phase is washed with water and the solvent removed to provide the mixture of condensation products as an oil.

In the fourth step of the present invention, the mixture of condensation products

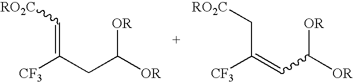

is cyclized to provide 4-trifluoromethyl-2(1H)-pyridinone.

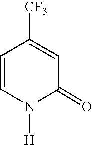

The cyclization may be accomplished by a variety of methods.

In a first method (a), the mixture of condensation products may be directly cyclized in one step to 4-trifluoromethyl-2(1H)-pyridinone by reaction with 1) an ammonium salt of an acid, preferably an organic acid, 2) formamide, or 3) formamide with an acid or acid salt. The ammonium salt of any aliphatic or aromatic organic acid can be used, but for convenience of processing, the ammonium salts of C₁-C₄ alkanoic acids are preferred. Ammonium formate and ammonium acetate are most preferred.

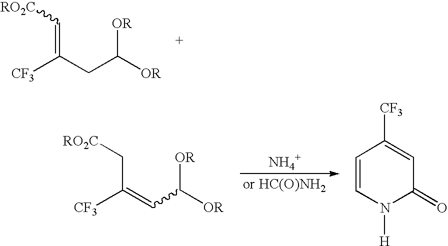

Approximately equimolar quantities of the mixture of condensation products and the ammonium salt of the organic acid are minimally required in the process, although 2-4 fold excesses of the ammonium salt of the organic acid are generally preferred.

This reaction is preferably conducted in the absence of a solvent, but a polar high-boiling solvent that is miscible with water can be used. If a solvent is used, preferred solvents include amides such as formamide, dimethyl formamide, dimethyl acetamide and 1-methyl-2-pyrrolidinone with formamide being particularly preferred.

The reaction is conducted at a temperature from about 120° C. to about 180° C. Temperatures from about 150° C. to about 160° C. are usually preferred.

The product is isolated by conventional techniques such as by precipitation followed by filtration.

In a typical reaction, the mixture of condensation products is dissolved in the polar aprotic solvent with the ammonium salt of the organic acid. The mixture is heated until the reaction is complete. After cooling to about 65° C., the mixture is diluted with water and, optionally, brine causing the product to precipitate. The 4-trifluoromethyl-2(1H)-pyridinone is isolated by filtration, washed with toluene, and dried.

In a second method (b), the mixture of condensation products may be treated with an unsubstituted amide (RCONH₂) and methoxide to form a mixture of amides which is subsequently cyclized with acid.

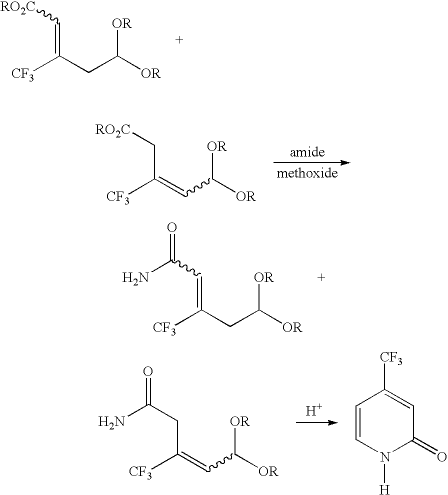

Any unsubstituted C₁-C₄ amides can be used, but formamide is preferred.

In the first step, approximately equimolar quantities of the mixture of condensation products, formamide and catalytic (e.g. 5%) methoxide (in methanol) are minimally required in the process, although an excess of formamide and 0.3-0.7 molar equivalents of methoxide (in methanol) is generally preferred.

This reaction is conveniently performed in the presence of a polar solvent that is miscible with water. Preferred solvents include amides such as formamide, dimethyl formamide, dimethyl acetamide and 1-methyl-2-pyrrolidinone, ethers such as tetrahydrofuran, and alcohols, preferably methanol.

The reaction is conducted at a temperature from about 40° C. to about 150° C. Temperatures from about 60° C. to about 100° C. are usually preferred.

The intermediate mixture of amides is cyclized without isolation by the addition of a protic inorganic or organic acid. Acetic acid and aqueous hydrochloric acid are particularly preferred. Enough acid to neutralize the base initially charged and an additional 0.05-0.20 molar equivalents is required.

In a typical reaction, the mixture of condensation products is dissolved in the polar aprotic solvent with the formamide. The alkoxide is added and the mixture is stirred at 25-40° C. temperature for 1-16 hours. The mixture is then optionally subjected to atmospheric distillation to remove volatile components. After cooling to ambient temperature, acetic acid is added and mixture is then subjected to atmospheric distillation until the pot temperature reaches about 145° C. and the overheads temperature reaches about 95° C. After cooling to ambient temperature, the 4-trifluoromethyl-2(1H)-pyridinone is isolated by standard procedures.

In a third method (c), the mixture of condensation products may be treated with a strong mineral acid and water to form a lactone which is subsequently reacted with ammonia.

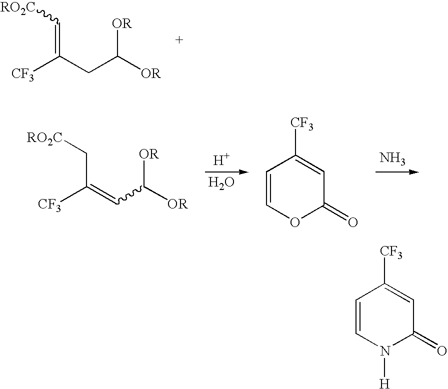

Any strong mineral acid can be used, but sulfuric acid is preferred.

In the first step, approximately equimolar quantities of the mixture of condensation products, acid and water are minimally required in the process, although an excess of water is generally preferred. This reaction is conveniently performed without any additional solvents at the reflux temperature of the mixture.

In a typical first step, the mixture of condensation products is added to water and acid and the mixture heated to reflux and distilled. The two phase distillate boiling from about 95-100° C. at atmospheric pressure is collected. The lower phase contains the intermediate lactone.

The intermediate lactone layer is then reacted with ammonia. The ammonia may be anhydrous or in solution with a solvent such as water, methanol or acetonitrile. While equimolar amounts of ammonia are minimally required, excesses are usually preferred. The reaction with ammonia is usually conducted below room temperature. With aqueous mixtures, the reaction is usually performed between about 0 and about 10° C. With anhydrous ammonia, the reaction is usually performed at about −41° C.

In a typical second step, the lactone layer is cooled to about 10° C. and vigorously stirred with additional water. Aqueous ammonia is then slowly added while maintaining the temperature below about 5° C. After warming to ambient temperature, the 4-trifluoromethyl-2(1H)-pyridinone is isolated by standard procedures.

In a fourth method (d), the mixture of condensation products may be treated with an organic anhydride and PPTS to form a mixture of dienoates which is subsequently cyclized with 1) an ammonium salt of an acid, preferably an organic acid, 2) formamide, or 3) formamide with an acid or acid salt as in method (a) or with an unsubstituted amide (RCONH₂) and methoxide to form a mixture of amides which is subsequently cyclized with acid as in method (b).

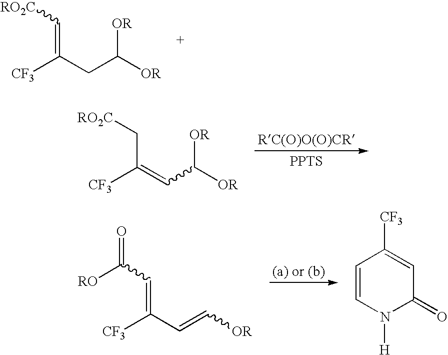

In the first step, approximately equimolar quantities of the mixture of condensation products and acetic anhydride are minimally required in the process, although an excess of acetic anhydride is generally preferred. Only a catalytic amount of PPTS is required, 1 to 2 mole percent usually being sufficient.

This reaction is conveniently performed without any additional solvents at the reflux temperature of the mixture.

In a typical first step, the mixture of condensation products, acetic anhydride and PPTS is stirred, heated to reflux and distilled at atmospheric pressure. After no more distillate is being collected and the pot temperature has reached about 145-150° C., the mixture is cooled to room temperature and the mixture of dienoates is isolated by standard procedures. The mixture of dienoates is then converted to 4-trifluoromethyl-2(1H)-pyridinone as described in the procedures of method (a) or method (b) above.

The following examples are presented to illustrate the invention.

### EXAMPLES

### Example 1

### Preparation of 4-Chloro-4-ethoxy-1,1,1-trifluoro-2-butanone

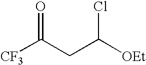

To a 100 mL three neck round bottom flask fitted with a thermocouple and a dry ice/acetone condenser was charged with 26.4 g (0.37 mol) of ethyl vinyl ether. The reaction vessel was then submerged into an ice-water bath for cooling. Then 49 g (0.37 mol) of trifluoroacetyl chloride was bubbled sub-surface through the reaction mixture. After completing the addition of the acid chloride, the ice-water bath was removed and the solution was allowed to warm to room temperature. The internal reaction temperature was not allowed to rise above 25° C. The reaction progress was monitored by GC. GC analysis indicated that the reaction mixture contained unreacted starting material. The reaction mixture was cooled in an ice-water bath and another 13 g (0.09 mol) trifluoroacetyl chloride was bubbled sub-surface through the reaction mixture. GC analysis indicated that the reaction still contained starting material so the process was repeated with another 19 g (0.14 mol) addition of trifluoroacetyl chloride. The reaction mixture was collected to give 58.6 g of product (˜94% crude yield and ˜71% pure by relative GC area) For 4-chloro-4-ethoxy-1,1,1-trifluoro-2-butanone: ¹H NMR (CDCl₃, 300 MHz), δ 1.25 (t, J=6 Hz, 3H), 3.38 (dd, J=18.0, 3.0 Hz, 1 H), 3.51 (dd, J=15.0, 9.0 Hz, 1 H), 3.63 (dq, J=9.0, 6.0 Hz, 1H), 3.98 (dq, J=9.0, 6.0 Hz, 1H), 5.97 (dd, J=6.0, 3.0 Hz, 1H). GCMS (PCI—NH3): m/z 204.0165.

### Example 2

### Preparation of 4-Chloro-4-ethoxy-1,1,1-trifluoro-2-butanone

To a 50 milliliter (mL) three neck round bottom flask fitted with a thermocouple and a dry ice/acetone condenser was charged 7.21 g (0.1 mol) of ethyl vinyl ether. The reaction vessel was then submerged into an ice-water bath and the internal reaction temperature was allowed to lower to about 4° C. Then 11.95 g (0.09 mol) of trifluoroacetyl chloride was bubbled sub-surface through the reaction mixture. After completing the addition of the acid chloride, the ice-water bath was removed and the solution was allowed to warm to room temperature. The internal reaction temperature was not allowed to rise above 25° C. The reaction progress was monitored by GC. Once the reaction was complete, GC analysis indicated the crude reaction mixture contained 4-chloro-4-ethoxy-1,1,1-trifluoro-2-butanone as the major product. GC analysis of this mixture was similar to that reported (for Example 1). This mixture was taken onto the mixture of acetals formation without any further isolation/purification.

### Example 3

### Preparation of 4-Chloro-4-ethoxy-1,1,1-trifluoro-2-butanone

A 500 mL jacketed reactor was equipped with a cooling bath and mechanical stirring. To this vessel, blanketed with nitrogen, was charged 95.81 g (1.33 mol) of ethyl vinyl ether in one portion. The circulation bath temperature was set at 0° C., mechanical stirring was turned on, and the reactor contents were allowed to cool down. Once the internal reaction temperature reached about 2° C., then 148.1 g (1.12 mol) of trifluoroacetyl chloride was slowly bubbled through the reaction mixture via a subsurface dip tube over a 2.5 h period. The internal reaction temperature was kept below 12° C. by adjusting the rate of gas addition. Once the trifluoroacetyl chloride addition was completed, the subsurface dip tube was removed from the vessel and the reaction mixture was allowed to stir with cooling for an additional 1 h 27 min. The reaction mixture was bottom drained from the vessel to afford 232.1 g of a colorless liquid. ¹⁹F NMR assay of this mixture (using 98% 2,4-dichlorobenzotrifluoride as an internal standard) indicated a 93% isolated yield and 92% purity for 4-chloro-4-ethoxy-1,1,1-trifluorobutan-2-one.

### Example 4

### Preparation of Mixture of Acetals

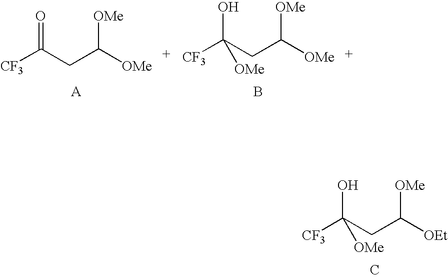

To 25 milliliter (mL) round bottom flask cooled in an ice-water bath and charged with 10 mL of methanol was added 10 g (0.05 mol) of neat 4-chloro-4-ethoxy-1,1,1-trifluoro-2-butanone via syringe over a 20 min period. The internal reaction temperature was maintained below 15° C. by the addition rate of the 4-chloro-4-ethoxy-1,1,1-trifluoro-2-butanone. The reaction mixture was then placed into a storage bottle and no further purification was performed. GC/EIMS analysis indicated a mixture of acetal A and hemi-ketals B and C. For acetal A: (relative intensity) m/z 185 (1), 155 (87), 75 (100), 69 (37). For hemi-ketal B: (relative intensity) m/z 217 (1), 186 (3), 155 (57), 75 (100), 69 (17). For hemi-ketal C: (relative intensity) m/z 231 (0.6), 201 (2), 169 (7), 155 (41), 89 (32), 75 (100), 61 (47).

### Example 5

### Preparation of Mixture of Acetals

To a three neck 100 mL round bottom flask equipped with a thermocouple, nitrogen padding, and an addition funnel was charged 35 mL of methanol and the reaction vessel was then cooled in an ice-water bath. To the methanol was con-added the 4-chloro-4-ethoxy-1,1,1-trifluoro-2-butanone (from Example 2) via addition funnel over a 19 min period. During the addition, the internal reaction temperature rose from 4° C. to 17° C. The reaction mixture was stirred for an additional 3 h and then allowed to stand overnight. This mixture was then taken into the next step without any further purification. GC of the acetal mixture was similar to that reported in example 4.

### Example 6

### Preparation of Mixture of Condensation Products

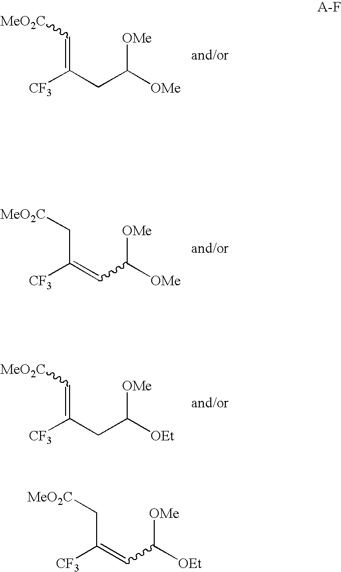

To a 50 milliliter (mL) three neck round bottom flask equipped with a dry-ice acetone condenser was charged with 5.72 g (0.08 mol) of ethyl vinyl ether and the mixture was cooled in an ice-water bath. To this cooled reaction mixture was added 12.2 g (0.09 mol) of trifluoroacetyl chloride (bubbled via sub-surface). After completing addition of the acid chloride, the reaction mixture was allowed to warm to ambient temperature and allowed to stir an additional 30 min. This mixture was then con-added over a 24 min period to an ice-water bath cooled 100 mL (milliliter) round bottom flask charged with 35 mL of methanol. The cold bath was removed and the solution allowed to stir an additional 1 h. The solution was allowed to stand overnight. The reaction mixture was then cooled in an ice-water bath, and to this mixture was slowly added 9.68 g (0.05 mol) of a 30% sodium methoxide in methanol solution. To this mixture was added 14.6 g (0.08 mol) of trimethylphosphonoacetate (TMPA) in one portion. To this mixture was then added 14.41 g (0.08 mol) of 30% sodium methoxide in methanol via addition funnel over a 9 min period. The cold bath was removed and the solution allowed to warm to ambient temperature and stirred an additional 1 h. The reaction mixture was concentrated on a rotovap at 30° C. until a slurry was observed. The residue was partitioned between 30 mL of hexanes and 20 mL of water. The bottom aqueous layer was extracted with 20 mL of hexanes. The combined hexane layers were concentrated on a rotovap at 30° C. to give 9.71 g of condensation products. No further purification was done on this mixture. GC/EIMS analysis indicated the presence of six peaks consisting of olefin products (A:B:C:D:E:F) in a relative ratio of (31:42:5:2:12:6). For olefin A: (relative intensity) m/z. 241 (0.20), 211 (15), 179 (70), 75 (100), 59 (5). For olefin B: (relative intensity) m/z 241 (1), 211 (100), 194 (11), 179 (51), 169 (35), 152 (11), 137 (15), 75 (24), 59 (54). For olefin C: (relative intensity) m/z 255 (0.03), 241 (0.30), 225 (7), 211 (13), 193 (2), 179 (53), 165 (31), 137 (8), 89 (57), 61 (100). For olefin D: (relative intensity) m/z 241 (0.80), 222 (4), 211 (100), 194 (8), 179 (40), 169 (30), 152 (12), 137 (17), 75 (46), 59 (72). For olefin E: (relative intensity) m/z 256 (0.5), 236 (4), 225 (49), 211 (72), 179 (65), 165 (93), 137 (24), 89 (19), 75 (100), 59 (42). For olefin F: (relative intensity) m/z 255 (1), 236 (6), 225 (88), 211 (43), 179 (29), 165 (100), 152 (20), 137 (33), 75 (87), 59 (35).

### Example 7

### Preparation of Mixture of Condensation Products

The acetal/methanolic mixture (Example 5) was chilled using an ice-water bath. To this mixture was con-added 21.1 g (0.12 mol) of 30% sodium methoxide in methanol via addition funnel. The addition rate was maintained to keep the internal reaction temperature below 20° C. After completing base addition, the solution pH was basic as measured by pH paper. To the reaction mixture was added 21.8 g (0.120 mol) of trimethylphosphonoacetate (TMPA) via syringe in one portion. The reaction mixture was allowed to cool down to about 2° C. using ice-water bath cooling and then 21.6 g (0.12 mol) of 30% sodium methoxide in methanol was con-added via addition funnel over 15 min. The internal reaction temperature rose from 2° C. to 8° C. during the base addition. The cold bath was removed and the solution was allowed to warm to ambient temperature with stirring for 2.5 h. The reaction mixture was concentrated on a rotovap and the residue was partitioned between 50 mL hexanes and 50 mL of water. The aqueous layer was separated and extracted with 30 mL of additional hexanes. The combined hexanes layers were then concentrated on a rotovap to give 22.72 g of a mixture of condensation products. This mixture was taken onto the next reaction step without further purification. GC analysis of this mixture of condensation products was similar to that reported for Example 6.

### Example 8

### Cyclization to 4-trifluoromethyl-2(1H)-pyridinone)

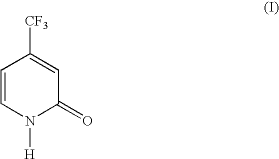

To a 100 mL three-neck round bottom flask equipped with a Dean-Stark trap, thermocouple, nitrogen sweep, and addition funnel was charged 13.4 g (0.173 mol) of ammonium acetate and 14 g (0.312 mol) of formamide. To the addition funnel was charged the mixture of condensation products (Example 7). The reaction mixture was heated to about 140° C. The condensation products were con-added through the addition funnel at a rate so that the internal reaction temperature did not drop below 130° C. The addition took 13 min to complete. The internal solution temperature was heated to 150-160° C. and then distillate was allowed to collect in the Dean-Stark trap. Two layers were observed in the trap, and the bottom layer was added back into the reaction pot. The distillation was allowed to proceed about 3 h 15 min until no more of the starting mixture of condensation products were present. The reaction vessel was cooled to 90° C. and then 20 mL of water was added to the mixture followed by 20 mL of a 10% aqueous brine. The reaction mixture was then cooled in an ice-water bath for 1 h at which time a precipitate formed. The slurry was suction filtered and the filter cake was washed with 15 mL of fresh water. The cake was air-dried in a hood overnight to afford 10.8 g (66% yield [based on starting ethyl vinyl ether molarity] and 97% purity by LC assay) of 4-trifluoromethyl-2(1H)-pyridinone as a light brown solid. mp (uncorrected) 151-157° C. (literature reported value 156-160° C.). ¹H NMR (CDCl₃, 300 MHz) δ 6.46 (dd, J=6, 3 Hz) 1H), 6.86 (bs, 1H), 7.52 (d, J=6 Hz), 1H), 13.5 (bs, 1H).

What is claimed is:

1. A process for the preparation of 4-trifluoromethyl-2(1H)-pyridinone (I), 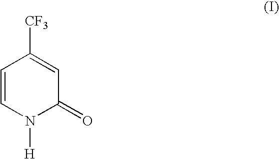
which comprises:
i) contacting an alkyl vinyl ether of the formula 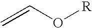
in which R represents a C₁-C₄ alkyl
with trifluoroacetyl chloride to provide a 4-chloro-4-alkoxy-1,1,1-trifluoro-2-butanone of the formula: 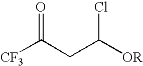
in which R is as previously defined;
ii) contacting the 4-chloro-4-alkoxy-1,1,1-trifluoro-2-butanone with a C₁-C₄ alcohol, to provide a mixture of acetals of the formula 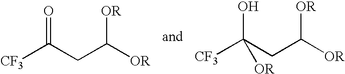
in which each R is independently as previously defined;
iii) contacting the mixture of acetals with a trialkyl phosphonoacetate of the formula: 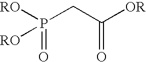
in which R is as previously defined,
in the presence of a base and an alcohol or glycol solvent to provide a mixture of condensation products of the formula 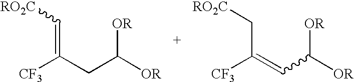
in which each R is independently as previously defined;
and
iv) cyclizing the mixture of condensation products to provide 4-trifluoromethyl-2(1H)-pyridinone.

2. The process of claim 1 in which the mixture of condensation products is cyclized by reacting with an ammonium salt of an organic acid and/or with a mineral acid and/or with formamide.

3. The process of claim 2 in which the ammonium salt of an organic acid is ammonium formate or ammonium acetate.

4. The process of claim 1 in which the mixture of condensation products is cyclized by reacting with an unsubstituted amide and methoxide to form a mixture of amides which is subsequently cyclized with acid.

5. The process of claim 1 in which the mixture of condensation products is cyclized by reacting with an acid and water to form the lactone which is subsequently reacted with ammonia.

6. The process of claim 1 in which the mixture of condensation products is cyclized by reacting with an organic anhydride and pyridinium p-toluenesulfonate to form a mixture of dieneoates which is subsequently cyclized by reacting with an ammonium salt of an organic acid or a mineral acid or with formamide or by reacting with an unsubstituted amide and methoxide to form a mixture of amides which is subsequently cyclized with acid.
